(19) United States
(12) Patent Application Publication
(10) Pub. No.: US 2016/0051591 A1
(43) Pub. Date: Feb. 25, 2016

(54) METHOD FOR THE SEPARATION OF FUNCTIONAL INGREDIENTS IN PLACENTA

(71) Applicants: Super Well Biotechnology Corporation, Taichung City (TW); Ultra-Microrigin Biomedical Technology Co., Ltd., Hsinchu County (TW)

(72) Inventors: Zer-Ran YU, Taichung City (TW); Be-Jen WANG, Taichung City (TW); Tsao-Chen WEI, Taichung City (TW); Hua-Ching LIN, Taichung City (TW); Ming-Hsi CHUANG, Hsinchu County (TW); Chu-Ting LIU, Hsinchu County (TW); I-Lung YU, Zhubei (TW); Chiu-Ying PENG, Hsinchu County (TW); Lin-Hsiang CHUANG, Hsinchu County (TW)

(21) Appl. No.: 14/702,077

(22) Filed: May 1, 2015

(30) Foreign Application Priority Data
Aug. 25, 2014 (TW) 103129255

(51) Int. Cl. A61K 35/50; B01D 11/02; A61K 31/565

(52) U.S. Cl. CPC A61K 35/50; A61K 31/565; B01D 11/02

(57) ABSTRACT

The present invention provides a method for the separation of placenta functional ingredients. By using the supercritical fluid technology, the placenta powder is placed inside an extraction tank, under the predetermined pressure and temperature; the supercritical CO₂ solvent is flown into the adsorption tank, in order to deodorize the fishy smell of the placenta powder, and extract the oil of the placenta powder. Under the same operating conditions as mentioned above, the deodorized and extracted placenta powder and supercritical CO₂/ethanol solvents are flown into an adsorption tank at the predetermined volumetric flow rate ratio to adsorb the estrogen of placenta powder to get the estrogen-removed placenta peptide extracts. Afterwards, supercritical CO₂/ethanol solvents are separated by rapid decompression to get the functional ingredients of placenta powder.

The current application claims a foreign priority to application number 103129255 filed on Aug. 25, 2014.

### BACKGROUND OF INVENTION

1. Field of the Invention

The present invention relates generally to the technology of separating active ingredients in placenta, and more particularly to a method which can separate estrogen, peptide and vitamins, trace elements and other functional ingredients in placenta.

2. Description of Related Art

As the source of nutrition for the fetus, placenta contains essential amino acids and pharmacologically active peptides, vitamins, minerals and various growth factors. According to research, its major functional ingredients include immunoglobulin, anti-aging peptides, rich lecithin, human brain phospholipids, lipopolysaccharides, a variety of vitamins and trace elements in addition to transfer factor, protein activity factor, collagen, and nucleic acid compounds. Therefore, placenta ingredients have the anti-aging, skin lightening, immunity improving and endocrine regulating functions. However, excessive use of estrogen, progesterone and other ingredients in placenta can cause cardiovascular diseases, cancer and other health problems.

Conventional methods of extraction and separation of the placenta peptide include the repeated freeze-thaw method, acid or alkaline hydrolysis, enzymatic method etc. For example, the disclosed US invention patent of US20130072466A1 proposes to use the mixed solvents of ethyl acetate, chloroform, ether, hexane and other organic solvents for the extraction of estrogen and other ingredients from placenta. The precipitation is then by adding alkaline substances in the extracted solution. Finally, the extracted substances are neutralized by acid solution. However, this process takes a long time and produces a large amount of acids, alkalis and organic solvents and wastes, resulting in the decomposition of functional ingredients and toxic residues. The Chinese patent of CN101837005B proposes to use alkali solution in the pretreatment of pig placenta and to hydrolyze the mixture by proteinase. The lipopolysaccharide and other functional ingredients in placenta are then precipitated by organic solvents. The US patent of US3041245 proposes to extract the albumin-containing active substance for skin care ingredients in mammalian placenta by using acidic solution with acetone organic solvent. The disclosed US invention patent US20130072466A1 proposes to use mixed organic solvents for the extraction of estrogen and other ingredients from placenta.

Regarding the above conventional extraction and separation methods, the processes are complicated and time-consuming with the addition of a large amount of acid solution, alkali solution, and organic solvents. The organic solvents may chemically react with the functional ingredients. During the solvent removal and condensation process after the extraction, the heating and vaporization of solvents may damage some of the functional ingredients and the solvents may residue in extracts or purified products. Therefore, such methods cannot easily, safely and efficiently separate and completely retain the functional ingredients in placenta including peptides, vitamins and trace elements.

### SUMMARY OF THE INVENTION

The main purpose of this invention is to provide a method to extract estrogen and oil from placenta without the complicated process of removing, condensation and separation process by using organic solvents. Moreover, it is free from the safety concern of solvent residues. It is low in cost can effectively improve the taste and nutrition of placenta powder products. Therefore, the method is a very environmentally friendly, safe, and practical one.

To achieve the above purposes, the present invention is a method for the extraction of functional ingredients in placenta. The steps include: deodorizing and extraction: under predetermined conditions of temperature and pressure, the placenta powder is placed inside an extraction tank before adding the supercritical solvents to deodorize the fishy smell of placenta powder and to extract placenta powder oil; adsorption of estrogen: under the same operating conditions as described in the above, the deodorized and extracted placenta powder and the supercritical CO₂/ethanol solvents are flown into an adsorption tank at the predetermined volumetric flow rate ratio to adsorb the placenta powder estrogen to get the estrogen-removed placenta extracts; separation: under the predetermined operating conditions of temperature and pressure, the supercritical solvents are separated by rapid decompression to get the functional ingredients of the placenta powder.

### BRIEF DESCRIPTION OF THE DRAWINGS

FIG. 1 is a flowchart illustrating the preferred implementation of the present invention.

FIG. 2 illustrates the oil yield of placenta powder in extraction tank of the preferred implementation of the present invention.

FIG. 3 illustrates the rejection of estrogen in adsorption tank of the preferred implementation of the present invention.

FIG. 4 illustrates the ADSC viability of the preferred implementation of the present invention.

FIG. 5 illustrates the extracted protein content analysis of the preferred implementation of the present invention.

FIG. 6 (a) illustrates the content analysis of protein of molecular weight of 58,000 of the preferred implementation of the present invention, (b) illustrates the content analysis of protein of molecular weight of 146,000 of the preferred implementation of the present invention.

FIG. 7 illustrates the extracted peptide content analysis of the preferred implementation of the present invention.

### DETAILED DESCRIPTION OF THE INVENTION

The method is elaborated on a preferred implementation of the present invention as well as drawings:

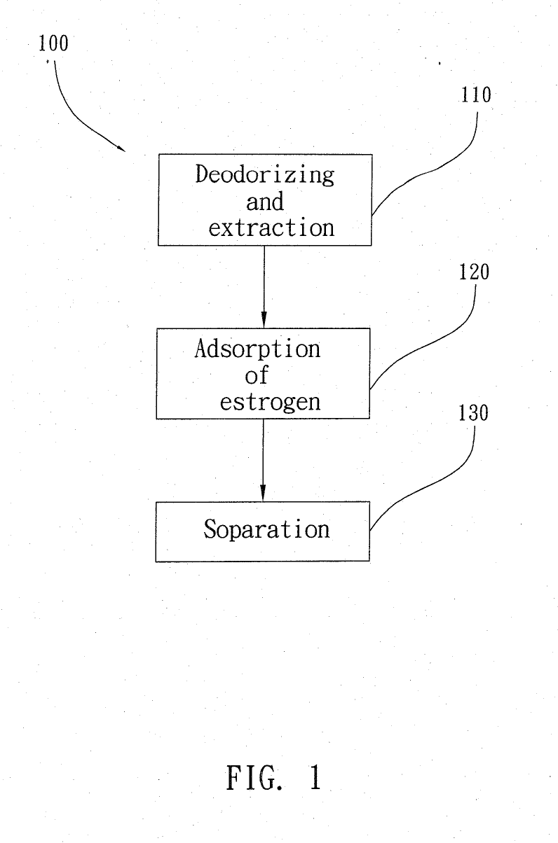

First, as shown in FIG. 1, in the preferred implementation of the present invention, the method for the separation of placenta functional ingredients 100 can deodorize the fishy smell of placenta powder and extract the placenta oil, estrogen, peptide and vitamins and trace elements. The first step is deodorizing and extraction 110: under the operating pressure of 2000-4000psi and temperature of 40-60° C., the 1-2 Kg placenta powder is placed in the extraction tank and extract the placenta oil and fishy odor by supercritical solvent for 2-3 hours. The oil will be collected at the bottom of the extraction tank. The placenta powder can be the dry powder of placenta of human, goat, pig, deer or other animals. The supercritical solvent is supercritical CO₂ solvent. The internal diameter of the stainless steel extraction tank is 60 mm and its height is 745 mm.

The second step is the adsorption of estrogen 120: under the same operating conditions (pressure 2000-4000 psi, temperature 40-60° C.)., the deodorized and oil-removal placenta powder, the supercritical CO₂/ethanol solvents at volumetric flow rate ratio of 15:1 is flown into the adsorption tank for 2-3 hours. The placenta powder estrogen will be absorbed by the adsorbents. The adsorption tank is a stainless steel tank of internal diameter at 60 mm and height at 450 mm filled with adsorbents such as silica gel, sephadex or resin.

The third step of the invention is separation 130: under the operating conditions of pressure 1000-1200 psi and temperature 40° C., the functional ingredients extracts from the placenta powder can be separated by rapidly pressure-decreased and collected at the bottom of the adsorption tank.

Under the different operating conditions (by changing pressure, temperature and volumetric flow rate ratio of solvents at the supercritical state), the following is the description of the methods analyze and quantify the samples of placenta powder, samples collected at the bottom of extraction tank and adsorption tank. The analysis of the samples are the following substances: (1) oil yield, (2) estrogen concentration, (3) adipose derived stem cells (ADSC) viability, (4) protein concentration, (5) protein electrophoresis, (6) peptide concentration.

The measurement of oil yield, represented by %, is to compute the quantity of oil collected at the bottom of the extraction tank to be compared with the quantity of the placenta powder placed in the extraction tank. The estrogen concentration is quantified by ELISA on progesterone and estriol. The estrogen rejection, represented by %, is to compute the estrogen concentration of the samples collected at the bottom of adsorption tank in comparison with the quantity of the placenta powder. The measurement of ADSC viability (%) of collecting the samples at the bottom of adsorption tank is analyzed and measured by cell flowmeter. The samples of protein concentration collected at the bottom of the adsorption tank, represented by mg/g dw, are measured by the Bradford reagent test. The samples collected at the bottom of the adsorption tank are also analyzed by SDS-PAGE protein gel electrophoresis, quantified and represented by mg/g dw. The samples of peptide concentration, represented by mg/g dw, is analyzed by HPLC quantitative analysis.

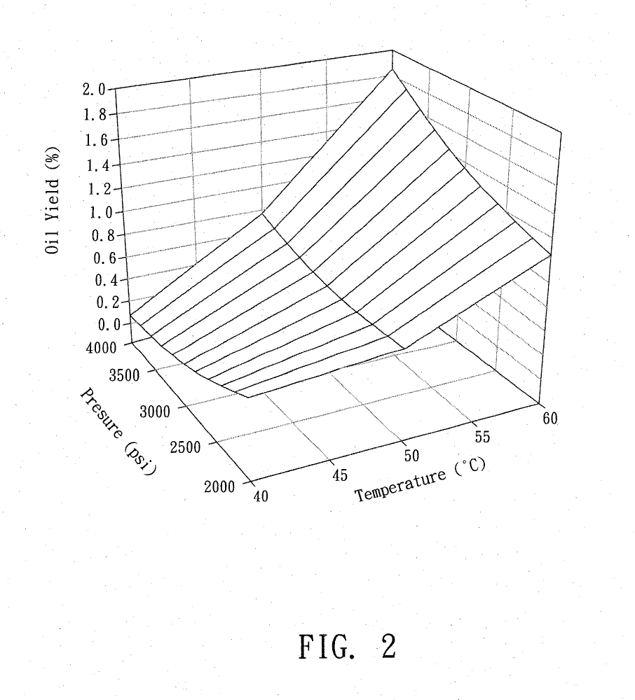

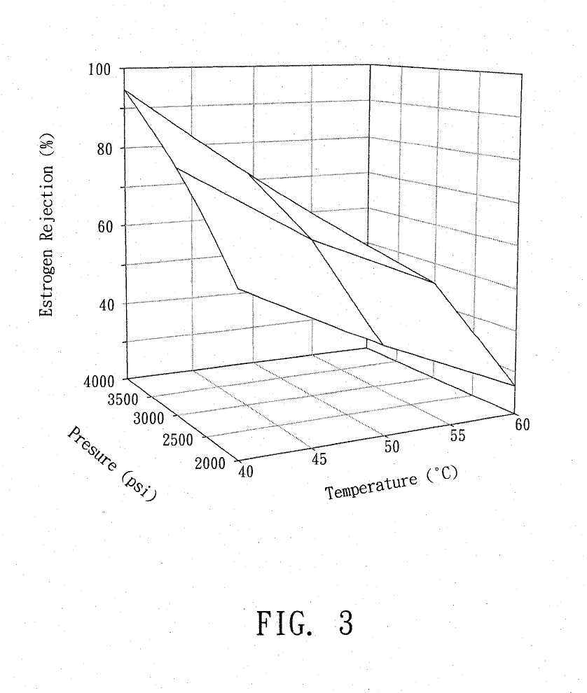

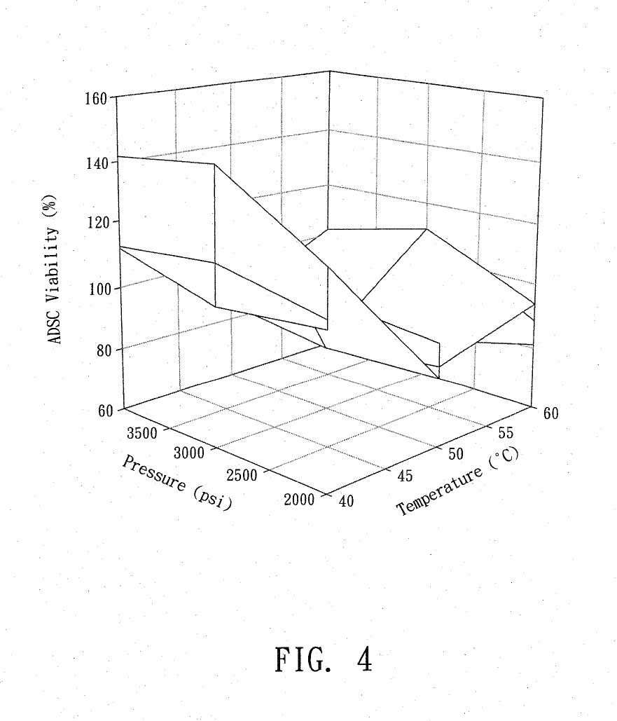

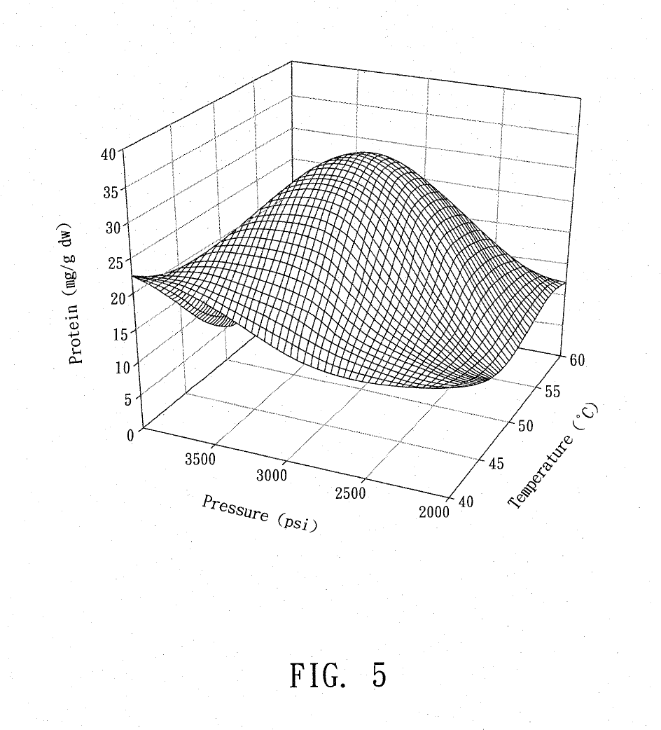

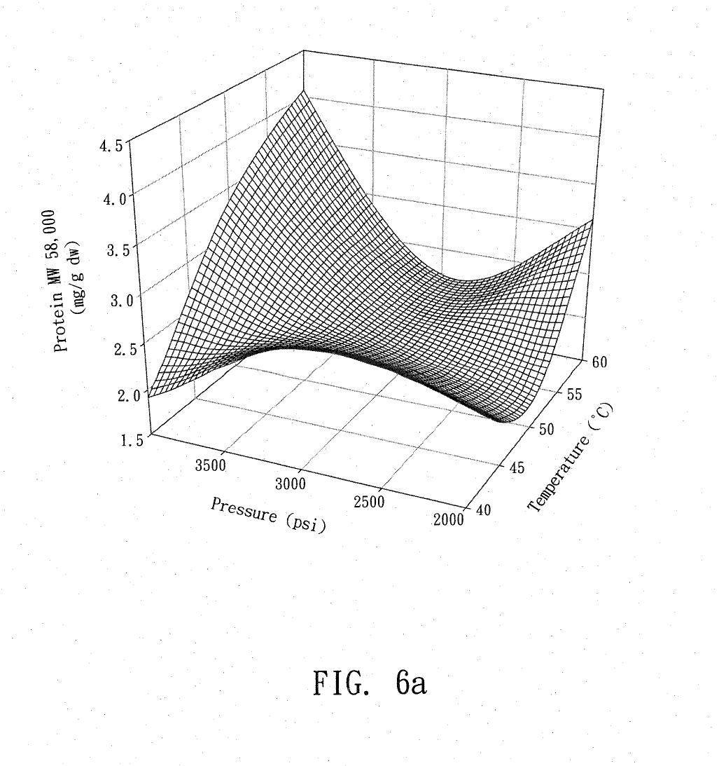

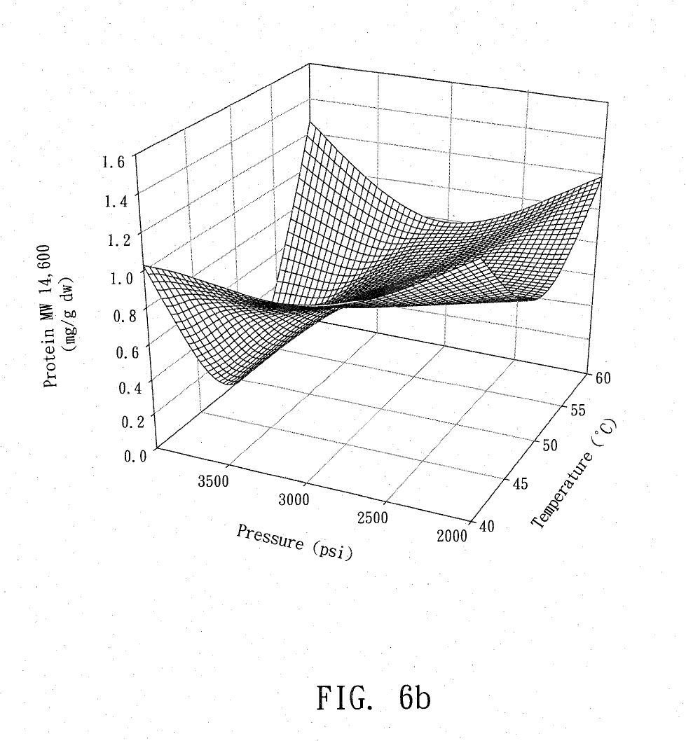

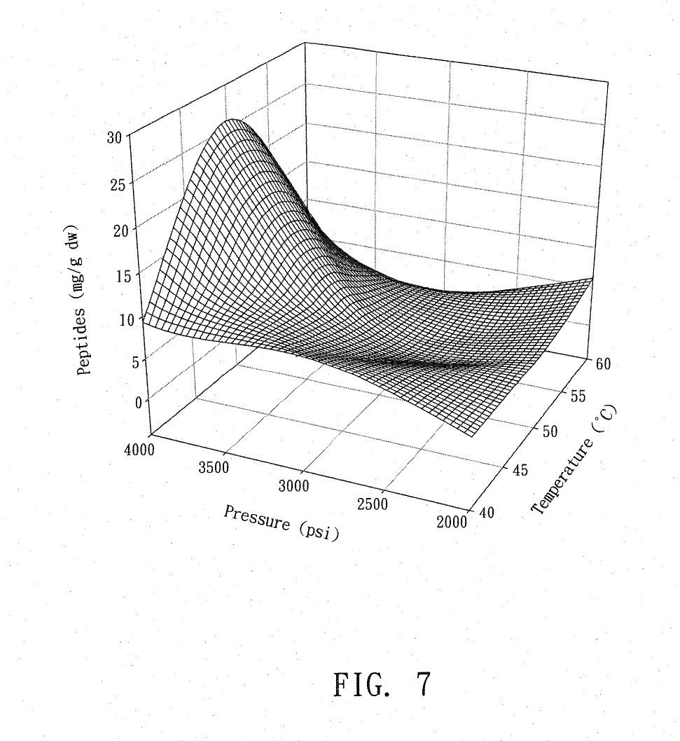

The experimental results can be divided into four parts: (1) oil yield optimal extraction conditions, (2) placenta estrogen optimal separation conditions, (3) selection of ADSC viability extraction conditions, (4) selection of extraction conditions for protein, peptide and active ingredients concentration. FIG. 2 curves represent the oil yield of placenta powder in extraction tank by using the solvents at the supercritical state under the operating conditions of pressure 2000-4000 psi and temperature 40-60° C. When the pressure increases from 2000 to 4000 psi and temperature increases from 40 to 60° C., oil yield efficiency increases significantly. With rising the supercritical state pressure and density, the oil solubility relatively increases. The rising temperature can increase the oil vapor pressure and the oil solubility will also increase accordingly. Therefore, FIG. 2 illustrates that the oil yield is optimal when the operating conditions are pressure 4000 psi and temperature 60° C. The data as shown in FIG. 3 suggest the extraction and separation efficiency of estrogen by SC-CO₂/ethanol solvents at volumetric flow rate ratio of 15:1 flown into the extraction tank under the operating conditions of pressure 2000-4000 Psi and temperature 40-60. The 6-7% ethanol is used as the co-solvent of the carbon dioxide at the supercritical state to promote the penetration of solvents at the supercritical state into the placenta powder to improve the estrogen extraction rate and separation effect. Therefore, FIG. 3 illustrates the high mass transfer and low viscosity effects of separation of 90-95% estrogen in placenta powder under the condition of pressure 4000 psi and temperature 40° C. Similar phenomenon can be found in the experimental results as shown in FIG. 4. The analysis results of the ADSC viability of the samples collected at the bottom of the adsorption tank suggest that solvents at the supercritical state under the operating conditions of pressure 4000 psi and temperature 40° C. for extraction and separation of the functional ingredients in placenta powder are most suitable for the promotion of adipose derived stem cells activation and regeneration capabilities. As shown in the experimental diagrams of FIG. 5, solvents at the supercritical state under the operating conditions of pressure 3000 Psi and temperature 50° C. can extract most protein from the placenta powder. By protein electrophoresis analysis of the samples extracted under these separation conditions for the comparison with the standard colloid maps of protein molecules of molecular weight ranging from 11,000 to 180,000, the results in the case of molecular weights of 14,600 and 58,000 are as shown in FIGS. 6 (a) and (b). Under the operating conditions of pressure 4000 psi and temperature 40° C. and 60° C., most protein functional ingredients can be extracted and separated from the placenta powder. By comparing the data as shown in FIGS. 5 and 6, the operating conditions of pressure 4000 psi and temperature 60° C. can facilitate the separation of the functional ingredients of proteins of molecular weight at 58,000. According to the experimental results of FIG. 7, the smaller molecular weights of peptides by HPLC analysis can be extracted and separated under the operating conditions of pressure 4000 psi.

The use of supercritical carbon dioxide, or supercritical carbon dioxide and ethanol as a solvent can result in high solubility and mass transfer rate. Moreover, low temperature, low viscosity and high density can promote the contact of the extracted functional ingredients and to speed up and selectively separate from the placenta powder functional ingredients such as estrogen, peptide and vitamins and trace elements. Therefore, the preparation method of the patent can avoid conventional shortcomings of using large amounts of organic solvents for the hydrolysis of estrogen component structures by using acid, alkali and enzyme solutions.

In addition, to compare the difference between the effect of the present invention and conventional dialysis extraction techniques, following experiments are conducted: (1) after physiological saline solution of 20 times the mass of the placenta powder is added for homogenization, the dialysis tube rejecting molecules of molecular weight of 14,000 is used for dialysis in RO water of five times homogenized solution for six hours to get the extraction samples; (2) by applying the proposed method to analyze the following physical properties of the extraction solutions obtained by using the two processing methods: (1) color, (2) fishy smell, (3) estrogen concentration, (4) ADSC viability, (5) protein concentration, (6) peptide concentration. The changes in color are measured by the Japan-made Denshoku Σ90 colorimeter and represented by Hunter L, a, b value. The fishy smell is measured by professionals and scored by 0 to 10 points. 10 points indicate the most acceptable sample and 0 point indicates the unacceptable sample.

The analysis of the experimental data results are as shown in Table 1. Table 1 suggests that the L value of the extracted sample by the proposed method is close to 94 as comparison of the placenta powder samples color L value 47. Hunter L can be represented by values ranging from 0 to 100. When the value is closer to 100, it means the degree of transparency and clarity is higher. When the Hunter a or b value is higher, it suggests that the color red or yellow concentration is higher, respectively. The placenta powder samples processed by dialysis are in dark yellow turbid state. Therefore, the placenta samples processed by the conventional dialysis techniques are unacceptable in appearance, color and clarity. The samples prepared by using the present method have no fishy smell, no estrogen and clear appearance. As shown in Table 1, the samples prepared by the present method have high protein content and peptide concentration as well as ADSC viability.

The present invention of the method for the separation placenta functional ingredients uses non-toxic solvents at the supercritical state coupled with physical deodorizing and extraction, adsorption and separation. Due to its high solubility, low viscosity and high mass transfer efficiency, the solvents at the supercritical state can speed up and selectively separate the functional ingredients such as estrogen, peptide, vitamins and trace elements from placenta powder without safety concerns of solvent residues. The CO₂ solvent at the supercritical state is an environmentally friendly and safe, and can be recycled and reused. Comparing to the conventional techniques of using a large amount of acid, alkali, enzyme solutions or organic solvents has the shortcoming of safety concern of residual solvents; the present invention is clearly progressive and has great practical value.

1. A method for the separation of functional ingredients in placenta by using the supercritical fluid technology to implement the following steps:
deodorizing and extraction: under the predetermined operating conditions of temperature and pressure, the placenta powder is placed inside an extraction tank before adding supercritical CO₂ solvent to deodorize the fishy smell of placenta powder and to extract oil from the placenta powder;
adsorption of estrogen: under the same operating conditions as described in the above, the deodorized and extracted placenta powder and supercritical CO₂/ethanol solvents are flown into an adsorption tank at the predetermined volumetric flow rate to adsorb the placenta powder estrogen to get the estrogen-removed placenta extracts; and
separation: under the predetermined operating conditions of temperature and pressure, supercritical solvents are separated by rapid decompression to get the functional ingredients of the placenta powder.

2. The method defined in claim 1, in the deodorizing and extraction step, placenta powder can be the dried placenta powder of human, sheep, pig, deer or other animal.

3. The method defined in claim 1, in the deodorizing and extraction step, under a pressure of 2000-4000 psi, temperature of 40-60° C., and placenta powder of 1-2 kg, the supercritical CO₂ solvent is flown into the extraction tank for 2-3 hours.

4. The method defined in claim 1, in the adsorption of estrogen step, under a pressure of 2000-4000 psi, and temperature of 40-60° C., the supercritical CO₂/ethanol solvent at volumetric flow ratio of 15:1 is flown into the adsorption tank for 2-3 hours.

5. The method defined in claim 1, in the separation step, under a pressure of 1000-1200 psi and temperature of 40° C., supercritical solvents are separated by rapid decompression.

6. The method defined in claim 3, the optimum operating conditions are: pressure of 4000 psi, temperature of 60° C., the oil yield is maximum.

7. The method defined in claim 4, when the optimum operating conditions are pressure of 4000 psi and temperature of 40° C., 90-95% estrogen of placenta powder be separated; the functional ingredients extracted from the placenta powder is most suitable for the promotion of adipose derived stem cells activation and regeneration.

8. The method defined in claim 4, when the optimum operating conditions are pressure of 3000 psi and temperature of 50° C., most proteins can be extracted from the placenta powder; when the operating conditions are pressure of 4000 psi, and temperature of 40° C. and 60° C., most content of protein functional ingredients can be extracted from the placenta powder.

9. The method defined in claim 1, said extraction tank is a stainless steel tank with an internal diameter of 60 mm and height of 745 mm.

10. The method defined in claim 1, said adsorption tank is a stainless steel tank with an internal diameter of 60 mm and height of 450 mm; it can be filled with the absorbents to adsorb estrogens.

11. The method defined in claim 9, said absorbent can be silica gel, sephadex or resin for adsorbing estrogen.

| | Dialysis | This Study |
|---|---|---|
| Color | | |
| L | 47.0 | 94.0 |
| a | 4.7 | −1.7 |
| b | 30.7 | 9.7 |
| Fishy Aroma | Strong | Weak |
| Estrogen Rejection (%) | 5 | 95 |
| ADSC Variability (%) | 102.0 | 142.0 |
| Protein (mg/g dw) | 1.8 | 35.8 |
| Peptide (mg/g dw) | 1.0 | 25.9 |
